(19) United States
(12) Patent Application Publication
(10) Pub. No.: US 2017/0119423 A1
(43) Pub. Date: May 4, 2017

(54) SURGICAL BLADE SYSTEM WITH POLISHED FINISH

(71) Applicant: Surgistar, Inc., Vista, CA (US)

(72) Inventors: Jonathan Woodward, Vista, CA (US); Payal Goel, Vista, CA (US)

(21) Appl. No.: 14/931,675

(22) Filed: Nov. 3, 2015

(51) Int. Cl. A61B 17/3211

(52) U.S. Cl. CPC A61B 17/3211; A61B 2090/3618

(57) ABSTRACT

A surgical blade includes a region that has a mirror finish such that the blade can reflect an image that can be viewed by a user. A first portion of the blade is a sharpened edge and a second portion of the blade has a mirror. The mirror portion of the blade can be useful in situations where visualization can be facilitated by using a mirror at or near the region where the blade is used. Upon identification or visualization of the region to be incised, a user can create an incision using the first, sharpened portion of the blade.

### BACKGROUND

Surgical blade systems are often used in regions of the anatomy where it can be difficult for a user to obtain an adequate view of the region where the blade is to be used. In some circumstances, the user may not have a direct line-of-sight to the anatomical region where the blade is to be used. This can make it difficult for the user to form a precisely located incision in tissue, which can be a major drawback in gaining proper access to a site that is to incised.

### SUMMARY

This disclosure relates to a feature of a scalpel for utility in any surgical procedure that requires access to regions of an anatomy that are difficult to view. Disclosed is a surgical blade that includes a region that has a mirror finish such that the blade can reflect an image that can be viewed by a user. In an embodiment, a first portion of the blade is a sharpened edge that is formed such as by a grinding or sharpening process. A second portion of the blade has a mirror finish that can be achieved, for example, by a polishing process. The mirror portion of the blade can be useful in situations where visualization can be facilitated by using a mirror at or near the region where the blade is used. Upon identification and/or visualization of the region to be incised, the user can create an incision using the first, sharpened portion of the blade.

In one aspect, there is disclosed a a surgical scalpel system, comprising a blade having a first region that forms a polished surface that reflect light in a manner that mirrors an image, the blade also having a second region that forms a sharpened edge that can cut through tissue.

In another aspect, there is disclosed a method of forming a surgical blade, comprising: forming a sharpened edge on a piece of material; and polishing a first region of the piece of material so that the first region includes a surface that reflects light in a manner that mirrors an image.

In another aspect, there is disclosed a method of cutting tissue, comprising: using mirrored surface of a blade to view a tissue site; and using a sharpened edge of the blade to incise tissue at the tissue site.

Other features and advantages should be apparent from the following description of various embodiments, which illustrate, by way of example, the principles of the invention.

### BRIEF DESCRIPTION OF THE DRAWINGS

FIG. 1A shows an example scalpel system.

FIG. 1B shows an exploded view of the scalpel system.

FIG. 2 shows a first view of a blade element of the scalpel system.

FIG. 3 shows a second view of a blade element of the scalpel system.

### DETAILED DESCRIPTION

Before the present subject matter is further described, it is to be understood that this subject matter described herein is not limited to particular embodiments described, as such may of course vary. It is also to be understood that the terminology used herein is for the purpose of describing a particular embodiment or embodiments only, and is not intended to be limiting. Unless defined otherwise, all technical terms used herein have the same meaning as commonly understood by one skilled in the art to which this subject matter belongs.

Disclosed is a surgical blade that includes a region that is polished to a mirror finish such that the blade can reflect an image that can be viewed by a user. In an embodiment, a first portion of the blade is a sharpened edge that is formed such as by a grinding process. A second portion of the blade has a mirror finish that can be achieved, for example, by a polishing process. In an embodiment, the scalpel system is configured for use in oral surgery for a human although the scalpel system can be adapted for use in other parts of human or other animal anatomy.

FIG. 1A shows a perspective view of a scalpel system having a handle 105 and a blade or scalpel 110 that is removably attachable to the handle 105. FIG. 1B shows the system with the scalpel 110 detached from the handle 105. The scalpel 110, which is described in further detail below, has a first region formed of at least one sharpened edge configured to cut through tissue, and a second region formed of a mirrored surface. The handle 105 has an attachment feature 115 where the scalpel 110 may be attached and removed from the handle. It should be appreciated that the particular shape of the handle 105 and the scalpel 110, as well as the overall shape of the scalpel system, may vary. Thus, the scalpel system shown in FIG. 1 is just an example and is not limiting to this disclosure. The attachment feature 115 may vary in structure and can be any type of attachment mechanism that removably attached a scalpel to a handle.

FIGS. 2-3 shows an exemplary scalpel 110. The scalpel 110 has a coupling region 205 that is configured to be attached to the handle 105. As mentioned, the mechanism of attachment to the handle may vary. In the illustrated embodiment, the coupling region 205 has an opening that is configured to mate with or otherwise couple to a complementary shaped coupling region of the handle 105. In the illustrated embodiment, the scalpel has an elongated shape although the shape may vary.

FIG. 2 shows a first side view of the blade or scalpel 110, which includes a planar first region 210 having a surface 212 with a mirror finish. That is, the surface 212 of the first region 210 is polished to a finish such that the first region reflects an image when positioned so as to face the image. The first region can be a flat or curved plane, concave, or convex, or a combination thereof, such that the first region reflects rays of light in a manner that generates a reflected image. The first region can also bent such that it includes a first portion that is flat and that lies along a first plane a second portion that is flat and that lies along a second plane that is non-parallel to the first plane.

The first region 210 may be located on a distal section of the scalpel 110. In an embodiment, only a portion of the scalpel 110 has a mirror finish. In another embodiment, the entire scalpel has a mirror finish. In another embodiment, the scalpel 110 has two or more side surfaces with one side surface being polished to a mirrored surface and at least one other side surface being not polished to a mirrored surface.

The mirror finish of the scalpel 110 can be achieved in a variety of manners, such as by polishing the first region 210, whether before or after the shape of the blade is created. The first region 210 or the entire scalpel 110 may be made of various materials, including stainless steel or any material that can be sharpened and made into a mirror.

With reference now to FIG. 3, the scalpel also includes a second region 215 that forms a sharpened edge 217. The sharpened edge of the second region 215 is sufficiently sharp to incise anatomical tissue. The sharpened edge can be immediately adjacent the mirrored first region 210 such that the edge forms an edge of the first region 210. In another embodiment, the sharpened edge is spaced apart from the first region 210.

Pursuant to a method of manufacture, the scalpel is machined out of a piece of suitable material, such as a metal. At least one edge is sharpened by a suitable method, such as by a grinding process for example. The mirrored surface is formed by polishing, or by another suitable method, at least a portion of the scalpel until the portion achieves a mirrored finish.

Pursuant to a method of use, a user identifies a site of anatomical tissue to be incised. In an embodiment, the user does not have a direct line of sight to the site of anatomical tissue to be incised and uses the mirrored first region to visualize the site. The site of anatomical tissue to be incised may be viewable only by using a mirrored surface in a non-limiting example.

The user uses the mirrored first region of the scalpel to visualize the site to be incised by reflecting at least a portion of the site off of the mirrored first region. In this manner, the user can view the site using the mirrored first region. The user then manipulates the scalpel to a position such that the sharpened edge of the scalpel can be used to incise the tissue. In an embodiment, the scalpel is shaped such that the mirrored first region reflects an image of the site while the user is incising tissue using the sharpened edge. The user can thus incise while also viewing an image of the site using the mirrored surface.

While this specification contains many specifics, these should not be construed as limitations on the scope of an invention that is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results.

Although embodiments of various methods and devices are described herein in detail with reference to certain versions, it should be appreciated that other versions, embodiments, methods of use, and combinations thereof are also possible. Therefore the spirit and scope of the appended claims should not be limited to the description of the embodiments contained herein.

1. A surgical scalpel system, comprising:
a blade having a first region that forms a polished surface that reflect light in a manner that mirrors an image, the blade also having a second region that forms a sharpened edge that can cut through tissue.

2. The surgical scalpel system of claim 1, wherein the first region is flat.

3. The surgical scalpel system of claim 1, wherein the first region is concave.

4. The surgical scalpel system of claim 1, wherein the first region is convex.

5. The surgical scalpel system of claim 1, wherein the first region is bent.

6. The surgical scalpel system of claim 1, wherein the blade is made of stainless steel, or any material that can be sharpened and made into a mirror.

7. The surgical scalpel system of claim 1, wherein the sharpened edge is an edge of the first region.

8. The surgical scalpel system of claim 1, further comprising a handle attached to the blade.

9. A method of forming a surgical blade, comprising:
forming a sharpened edge on a piece of material;
polishing a first region of the piece of material so that the first region includes a surface that reflects light in a manner that mirrors an image.

10. The method of claim 9, wherein the sharpened edge is an edge of the first region of the blade.

11. The method of claim 9, wherein polishing a first region comprises polishing a concave surface.

12. The method of claim 9, wherein polishing a first region comprises polishing a convex surface.

13. The method of claim 9, wherein polishing a first region comprises polishing a flat surface.

14. A method of cutting tissue, comprising:
using mirrored surface of a blade to view a tissue site; and
using a sharpened edge of the blade to incise tissue at the tissue site.

15. The method of claim 13, wherein the tissue site is not directly viewable without use of a mirrored surface.
